(19) United States
(12) Patent Application Publication
(10) Pub. No.: US 2025/0046410 A1
(43) Pub. Date: Feb. 6, 2025

(54) Method for generating quantitative distributions of health factors from patient graphs

(71) Applicant: Anjun Chen, Palo Alto, CA (US)

(72) Inventor: Anjun Chen, Palo Alto, CA (US)

(73) Assignee: ELHS Institute Inc., Palo Alto, CA (US)

(21) Appl. No.: 18/792,500

(22) Filed: Aug. 1, 2024

Related U.S. Application Data
(60) Provisional application No. 63/530,473, filed on Aug. 3, 2023.

(51) Int. Cl. G16H 10/60

(52) U.S. Cl. CPC G16H 10/60

(57) ABSTRACT

A method, process, and system include integrating patient records from various sources into patient health records, selecting target and background patients from the records, collecting standardized health factor data, constructing patient graphs with patient nodes connected to health factor nodes, measuring factor node connection characteristics, calculating the connection delta ratio for each factor, and generating a quantitative health factor distribution for the target sorted by the connection delta ratio.

### BACKGROUND

### Field of the Invention

The present invention relates to a novel method for generating quantitative health factor distribution for any target, such as disease or condition, from patient health records (PHR), including electronic health records (EHR) data. More particularly, the invention is directed to a method that extracts standardized health factors of target patients with a specific target and background patients without the target from PHRs, construct patient health factor graphs, and generates a quantitative distribution of health factors for the target from these graphs.

### Description of the Related Art

Health factors can be any clinical, biological, genetic, environmental, or social factors that may affect health conditions or diseases. A health factor that negatively affects health is considered a risk factor. Risk factors are usually determined individually by conducting clinical trials using association study or case-control study methods. Due to variations in different studies settings, different risk factors quantified in different studies for one specific disease are difficult to consolidate into a single quantitative profile or distribution.

A genome-wide association study (GWAS) can measure and analyze a large number of genetic factors simultaneously in the same experiment, generating a quantified profile of genetic factors concerning a phenotype. However, this method is limited to genetic factors, or at most biological factors, excluding other types of health factors.

Hospital EHR system and other health information systems record all types of health factors in large numbers for most diseases and conditions. Machine learning methods have been used to identify risk factors from EHR data. Patient knowledge graphs have been constructed from EHR data to predict diagnoses from symptoms. But no invention to date has utilized real-world EHR or PHR data to construct patient graphs to generate quantitative health factor distributions for diseases or conditions.

### REFERENCES

### Patent Citations

U.S. Pat. No. 10,839,962B2 2016-09-262020-11-17 International Business Machines Corporation: System, method and computer program product for evaluation and identification of risk factor.
U.S. Pat. No. 11,056,218B2 2016-05-312021-07-06 International Business Machines Corporation: Identifying personalized time-varying predictive patterns of risk factors.

### Non-Patent Citations

Stampfer MJ, Ridker PM, Dzau VJ. Risk factor criteria. Circulation. 2004;109(25 Suppl 1):IV3-5. doi:10.1161/01.CIR.0000133446.69171.7d.
Zhu Z, Zheng Z, Zhang F, Wu Y, Trzaskowski M, Maier R, Robinson MR, McGrath JJ, Visscher PM, Wray NR, Yang J. Causal associations between risk factors and common diseases inferred from GWAS summary data. Nat Commun. 2018 Jan 15;9(1):224. doi:10.1038/s41467-017-02317-2.
M. Rotmensch, Y. Halpern, A. Tlimat, et al. Learning a Health Knowledge Graph from Electronic Medical Records. Sci Rep 7, 5994 (2017). doi: 10.1038/s41598-017-05778-z.
Chen A. A novel graph methodology for analyzing disease risk factor distribution using synthetic patient data. Healthcare Analytics. 2022;2:100084. doi:10.1016/j.health.2022.100084.
Chen A, Huang R, Wu E, et al. The Generation of a Lung Cancer Health Factor Distribution Using Patient Graphs Constructed From Electronic Medical Records: Retrospective Study. J Med Internet Res. 2022;24(11):e40361. doi:10.2196/40361.
Chen A, Lu R, Han R, Huang R, Qin G, Wen J, Li Q, Zhang Z, Jiang W. Building practical risk prediction models for nasopharyngeal carcinoma screening with patient graph analysis and machine learning. Cancer Epidemiol Biomarkers Prev. 2022:EPI-22-0792. doi:10.1158/1055-9965.EPI-22-0792.

### SUMMARY OF THE INVENTION

In one embodiment of the present invention, patient health records are integrated to collect health factors for a disease or condition. These factors are used to construct patient graphs, from which health factor node connection delta ratios are calculated to generate a quantitative distribution of health factors for the disease or condition. In another embodiment, patient graphs are constructed for any target using provided patient health records. Connection delta ratios are then calculated to generate a quantitative health factor distribution with regard to the target.

### BRIEF DESCRIPTION OF THE DRAWINGS

The present invention can be better understood through the following detailed description of the exemplary embodiments, with reference to the accompanying drawings, in which:

FIG. 1 illustrates an example flowchart of the invented method for generating a quantitative distribution of disease health factors from PHR data, achieved through data standardization, patient graphs and quantification of factor node connections.

FIG. 2 presents an example block diagram of a patient graph connecting N number of patients to X number of health factors with each patient associated with one or multiple health factors.

FIG. 3 displays an example patient graph, consisting of patient nodes (small gray dots) connected to five health factors (big black dots), constructed using patient data from a disease patient graph study.

FIG. 4 showcases an example of quantitative health factor distribution for a disease, sorted by the Connection Delta Ratio (CDR) in descending order. The x-axis is labeled with health factor codes.

### DETAILED DESCRIPTION OF THE INVENTION

The following descriptions of various embodiments have been presented for the purpose of illustrating the invention. They are not intended to be exhaustive or limited to the embodiments disclosed. Many modifications and variations will be apparent, all without departing from the scope and spirit of the described embodiments. The flowchart, block diagrams, and graphs in the figures illustrate the architecture, functionality, and operation of possible implementations of systems, methods, and processes according to various embodiments of the present invention.

The terminology used herein is for the purpose of explaining the principles of the embodiments and is not intended to limit the present invention. Definitions of key terms are as follows:
PHRs: PHRs refer to the integrated patient health records containing data from EHR systems and other health information systems (HIS), genetic information, and biological information.
Target: A target refers to any health-related event including, but not limited to, a diagnosis of disease or condition; prescriptions of medications, procedures, treatments, preventions; prognosis of disease, condition, or general health.
Health factor: A health factor refers to any factors related to the target found in the PHRs including, but not limited to, any clinical factor, history factor, family factor, social factor, financial factor, environmental factor, physical factor, biological factor, and genetic factor.
Patient graph: A patient graph refers to a graph consisting of patient nodes connected to patients' health factor nodes. Patients include target patients with the target and background patients without the target.
CDR: CDR, or Connection Delta Ratio, is calculated from the health factor node connection characteristics for the target patients compared to those for the background patients. CDR represents a simple measure of the relative strength or importance for any given health factor with respect to the target.
Quantitative health factor distribution: A quantitative health factor distribution refers to a distribution of health factors by the CDR values. Because the clinical meaning of a health factor may vary for different targets, a distribution of health factors is specific to a target.

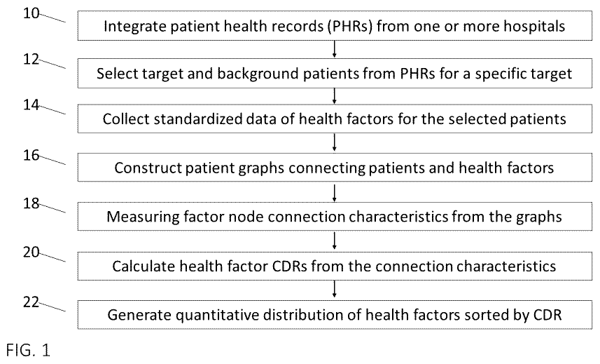

The present invention is described by referencing the appended figures representing preferred embodiments. FIG. 1 is an example flowchart of the invented method, process and system, which includes the following steps: integrating patient records into PHRs 10, selecting target and background patients from the PHRs 12, collecting standardized health factor data from the selected patients 14, constructing patient graphs with patient nodes connected to health factor nodes 16, measuring factor node connection characteristics for each factor 18, calculating the connection delta ratio (CDR) for each factor 20, and generating quantitative health factor distribution sorted by CDR value 22.

PHRs 10 are the source of patient health records integrated from EHR and other health information systems. Each patient's data may exist in EHR records, lab test records, pathology records, imaging records, pharmacy records, payment records, insurance records, and any other information recorded in health care. It may also include genetic tests and biological tests, as well as patient-generated health data. PHRs can be from a single hospital or a network of hospitals and care providers.

An equal number of target patients with the target disease and background patients without the disease are selected from the PHRs 12. Target patient selection uses diagnosis codes or terms, or any other criteria that can identify the disease. Background patients should be randomly selected to avoid patient bias in the control group. Additional exclusion or inclusion criteria, such as age and gender, may be applied to further restrict the patient groups.

Standardized data are collected from the selected patients' PHRs for the health factors 14. Each health factor is standardized as a unique concept within the data set, which can have different expressions or terms (i.e., synonyms) in the original health records. The types of health factors include, but are not limited to, condition, symptom, physical exam, observation, history, lab test, imaging result, procedure, medication, treatment, and other health data types such as demographics, survey, vital sign, and immunization.

For data collection, two different strategies can be used. In a preferred embodiment of element 14, a data-driven approach is used, which collects all health factors available in the patient records, i.e., does not limit to only known risk factors for the target disease or condition. As a result, unknown factors and previously overlooked factors will be collected into the dataset for patient graphs, which is required for identifying new health factors that may affect the disease.

In another embodiment of element 14, a knowledge-based approach is used, which selects only a set of known health factors from the patient records. For example, data for the known risk factors of the disease are collected into the dataset for graphs.

The patient health factor data are used to construct patient graphs 16. A patient graph is composed of patient nodes, health factor nodes, and relations or connections between the patient nodes and the factor nodes. An equal number of target patients with the disease and background patients without the disease should be included for calculating the connection delta ratio for each factor.

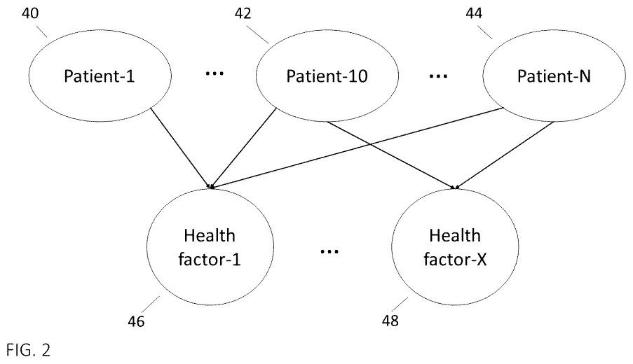

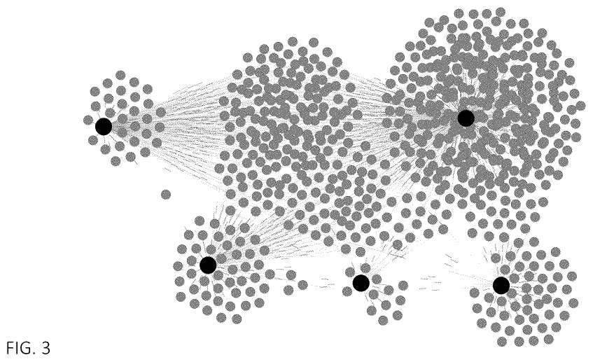

FIG. 2 is a block diagram of an example patient graph consisting of an N number of patients and an X number of health factors. One health factor may be shared by multiple patients, and one patient may have multiple factors. For example, health factor-1 46 is shared by 3 patients (patient-1 40, patient-10 42 and patient-N 44) in FIG. 2, while health factor-X 48 exists in 2 patients (patient-10 42 and patient-N 44). FIG. 3 is an example patient graph consisting of patient nodes (small gray dot) connected to 5 health factors (big black dot) constructed with patient data from a disease patient graph study. Lines represent relations between a factor and the patients having the factor.

CDR is designed as a simple measure of the relative strength or importance of any given health factor with respect to the target disease. From the patient graphs, different approaches can be used to measure factor node connection characteristics 18 and calculate health factor CDRs based on the connection characteristics 20.

In a preferred embodiment, the connection characteristics for each health factor node in element 18 are measured by the number of connections going from the factor node to the patient nodes. Connections to target patients are counted as target patient factor connections (TPC), and connections to background patients as background patient connections (BPC). The CDR in element 20 is defined and calculated by equation: CDR=(TPC−BPC)/(TPC+BPC).

In another embodiment, the connection characteristics for each factor node in element 18 are measured by the embedding of the factor nodes. The factor node embedding captures the connection topology around the factor node. Graph embedding can be calculated in different ways including, but not limited to, the embedding functions provided by the graph database storing the data. For example, after the patient health factor data are loaded into Neo4j graph database, a target patient graph can be constructed, in which the embedding for each factor node can be calculated by Neo4j as TPC. Similarly, a background patient graph can be constructed, in which the embedding for each factor node can be calculated by Neo4j as BPC. The embedding data can be exported to a file. The CDR in element 20 can be calculated by the same (TPC−BPC)/(TPC+BPC) formula or a different formula.

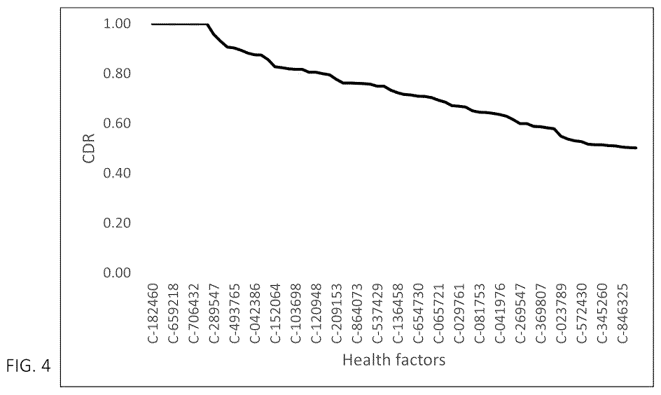

A quantitative health factor distribution 22 is generated by sorting the health factors by the CDR values. Depending on the applications, the distribution can be sorted in different ways such as in descending or ascending order, or the health factors are first categorized and then sorted within each category in descending or ascending order. Because the clinical meaning of a health factor may differ for different targets, a distribution of health factors is specific to a target, such as disease or condition. FIG. 4 is an example of quantitative health factor distribution sorted by CDR in descending order for a disease. The health factors shown on the x-axis have positive CDR values, indicating that they negatively affect the disease as potential risk factors.

A quantified distribution of health factors for a disease or condition has many applications. It helps identify new risk factors and foster a more complete understanding of all the factors affecting the disease or condition. In addition, a quantitative health factor distribution can be generated by the present invention for any health-related targets, potentially resulting in a broader understanding and improvement in health care, health living, and public health. The quantified distribution can also serve as a new source of prioritized health factors for feature selection in developing machine learning models for any health-related target.

What is claimed is:

1. A method comprising:
integrating patient health records (PHRs) from electronic health record systems, any other health information systems, biological tests, genetic tests, and patient-generated health data in one or more hospitals and health care organizations;
selecting an equal number of target patients with a disease and condition (DAC) and background patients without the DAC;
collecting standardized data of any number of health factors of the selected patients in the PHRs;
constructing patient graphs connecting patient nodes to patients' health factor nodes;
measuring each health factor node's connection characteristics for the target patients (TPC) and the background patients (BPC) separately;
calculating the connection delta ratio (CDR) using each health factor's TPC and BPC to provide a relative measure of the importance of a health factor for the DAC; and
sorting the health factors by CDR to generate a quantitative distribution of health factors specific to the DAC.

2. The method of claim 1, wherein the collected health factors include all available health factors in the PHRs.

3. The method of claim 1, wherein the measurement of patient health factor connection characteristics TPC and BPC is based on the count of connections from the factor node to the target or background patient nodes.

4. The method of claim 1, wherein the measurement of patient health factor connection characteristics TPC and BPC is based on the factor node embedding in the target patient graph or the background patient graph.

5. The method of claim 1, wherein the CDR is calculated using the equation: CDR=(TPC−BPC)/(TPC+BPC).

6. A computer-implemented method comprising:
defining a target of any health-related event including but not limited to disease or condition diagnosis, medications, procedures, treatments, preventions, and prognosis;
constructing patient graphs with patient nodes connected to patients' health factor nodes using patient health factor data collected for the target;
measuring each health factor node's connection characteristics for target patients (TPC) and background patients (BPC) separately;
calculating connection delta ratio (CDR) using each health factor's TPC and BPC to provide a relative measure of the importance of a health factor for the target; and
sorting the health factors by CDR to generate a quantitative distribution of health factors specific to the target.

7. The method of claim 6, wherein constructing patient graphs comprises:
an equal number of target patients with the target and background patients without the target; and
the health factors including any number of health factors related to the target found in patient health records integrated from different sources.

8. The method of claim 6, wherein measuring patient health factor connection characteristics TPC and BPC is based on the count of connections from the factor node to the target or background patient nodes.

9. The method of claim 6, wherein measuring patient health factor connection characteristics TPC and BPC is based on the factor node embedding in the target patient graph or the background patient graph.

10. The method of claim 6, wherein calculating CDR uses equation: CDR=(TPC−BPC)/(TPC+BPC).
